(19) United States
(12) Patent Application Publication
(10) Pub. No.: US 2018/0214628 A1
(43) Pub. Date: Aug. 2, 2018

(54) NASAL EJECTING CATHETER FOR HOME REMEDY OF NASAL IRRIGATION TREATMENT

(71) Applicant: Patent Wide International Corporation, Kaohsiung City (TW)

(72) Inventors: Lih-Chiu WU, Kaohsiung City (TW); Hui-Shuan LEE, Kaohsiung City (TW); Tze-Yu LEE, Kaohsiung City (TW)

(21) Appl. No.: 15/940,196

(22) Filed: Mar. 29, 2018

Related U.S. Application Data
(63) Continuation-in-part of application No. 13/592,919, filed on Aug. 23, 2012.

(51) Int. Cl. A61M 3/02; A61L 29/06; A61L 29/16; A61L 29/04

(52) U.S. Cl. CPC A61M 3/0279; A61L 29/042; A61L 29/16; A61L 29/06

(57) ABSTRACT

A nasal ejecting catheter for home remedy of nasal irrigation treatment comprises a catheter unit and a connecting unit. The catheter unit comprises an open end, a closed end on the opposite side and a plurality of apertures formed near the closed end. The connection unit comprises a connection portion for connecting the open end of the catheter and an assembling portion for connecting an injection unit. The catheter body is made of silicone, latex, thermoplastic elastomer or other soft material to be operated by the patient. The total area of apertures is smaller than the cross section area of inner lumen of the catheter in order to eject substantially even strength multiple thin spouts.

### RELATED APPLICATION

This application is a Continuation-In-Part of currently pending U.S. patent application Ser. No. 13/592,919 filed on Aug. 23, 2012.

### BACKGROUND OF THE INVENTION

### (a) Field of the Invention

The present invention relates to a nasal flushing device, and more particularly to a portable nasal flushing device.

### (b) Description of the Related Art

Recently, with climate change and over development, air population becomes a serious issue in our daily life. According to Medscape website (http://emedicine.medscape.com/article/232791-overview#a0156) in USA, every 1000 people, there are 146 people have chronic nasosinusitis, and estimated about 18 to 22 million doctor visits and 3.4 to 5.0 billion direct medical expense. However, the numbers still increase every year. So far, according to Medscape and Mayo Clinic websites, there is no cure for chronic nasosinusitis. The main symptoms are nasal obstruction, post nasal drip and cough. Surgical operation is only suitable for some cases, while antibiotics are only effective for acute bacterial infection rather than chronic symptoms. Therefore, all experts recommend home remedy of moistening the nasal cavity every day.

Please refer to FIG. 1. On each side, the nasal cavity proper between the nasal vestibule D in front and nasopharynx E on back is divided by three slightly curved plate like structures, the upper turbinate A, the middle turbinate B, and the inferior turbinate C into three slit like air passages, the upper nasal meatus A1, the middle nasal meatus B1 and the lower nasal meatus C1. All these structures and air passages are smaller in upper and larger in lower. All secretion from the paranasal sinuses is discharged from the several openings located in the roof upper nasal meatus A1 and the middle nasal meatus B1. Therefore, thick tenacious mucus generated by chronic nasosinusitis builds up from the narrow roof of the upper nasal meatus A1 and the middle nasal meatus B1 and spreads downward. The thick mucus is difficult to completed expelled even by forceful blowing nose and is easily accumulated within the nasal cavity. The mucus is inevitable dried by the respiratory air to become crust. Repeated coating of layers of crust on the nasal turbinated and accumulation of mucus in the nasal meatus causes obstruction of nasal passage known as nasal obstruction.

Among the methods of moistening the nasal cavity, nasal irrigation treatment using neti pot or other squeezable device has been considered the best of choice. All the nasal irrigation devices on the market are quite similar among each other.

For example, please refer to FIG. 2. As Taiwan patent no. M418689 which discloses a nasal irrigation device having: an applying portion 10 and a nozzle 20. The applying portion 10 has an inner space 11 and the nozzle 20 has an opening 21 as well as an extending tube 22 reaching the inner space 11 of the applying portion 10. When the inner space 11 is filled with water and compressed, the water is squeezed to the extending tube 22 and exit from the opening 21.

As previous description, for chronic sinusitis patient, the most desired locations to be moisturized are the upper part of upper nasal meatus A1 and the middle nasal meatus B1. The large nozzle 20 of the above mentioned nasal irrigation device can only be placed in the nasal vestibule; it can only produce a front to back single directional spout from the nasal vestibule. This spout can only pass through the wide inferior nasal meatus and cannot reach the narrow or obstructed upper part of upper nasal meatus A1 and the middle nasal meatus B1 to treat the symptoms correctly.

Moreover, the spout produced by the device might be too strong for nasal and nasopharyngeal mucosa or even choke the patient.

Therefore, it is desirable to provide a nasal flushing device to mitigate and/or obviate the aforementioned problems.

### SUMMARY OF THE INVENTION

An objective of the present invention is to provide an effective nasal flushing device which can easily be navigated along the tortuous nasal cavity for home remedy of nasal irrigation treatment.

In order to achieve the above mentioned objective, the nasal flushing device includes a catheter unit, a connecting unit and an injection unit.

The catheter unit comprises a catheter body having an open end and a closed end opposite the open end as well as a plurality of apertures formed near the closed end thereof and the total area of apertures is smaller than the cross section area of inner lumen of the catheter.

The connecting unit has a connecting portion for connecting the open end of the catheter and an assembling portion for connecting the injection unit.

The injection unit has a detachable syringe and a plunger provided in the syringe.

The nasal flushing device utilizes the catheter unit, the connecting unit, and the injection unit to deeply rinse the nasal cavity.

Other objects, advantages, and novel features of the invention will become more apparent from the following detailed description when taken in conjunction with the accompanying drawings.

### BRIEF DESCRIPTION OF THE DRAWINGS

FIG. 1 is side view of the nasal cavity.

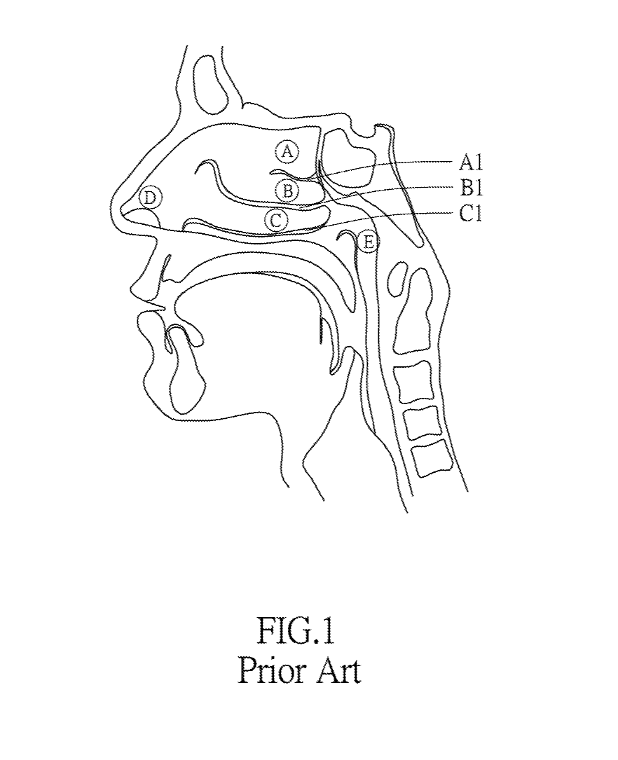

FIG. 2 is a cross-sectional view of a nasal irrigation device disclosed in Taiwan patent no. M418689.

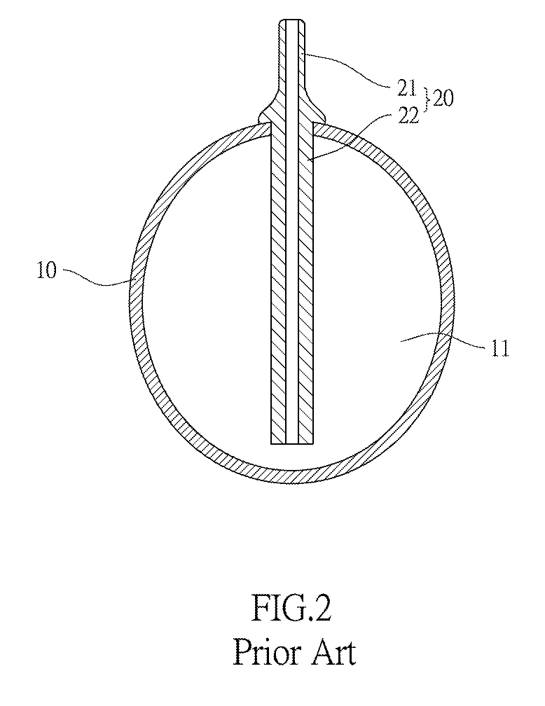

FIG. 3 is a perspective view of a nasal flushing device according to a first embodiment of the present invention.

FIG. 4 is a schematic drawing showing the nasal flushing device of the first embodiment being applied to the nasal cavity.

FIG. 5 is another schematic frontal view showing the nasal flushing device of the first embodiment being applied to the nasal cavity.

FIG. 6 is a perspective view of a nasal flushing device showing the TAA smaller than the CSA.

### DETAILED DESCRIPTION OF THE PREFERRED EMBODIMENTS

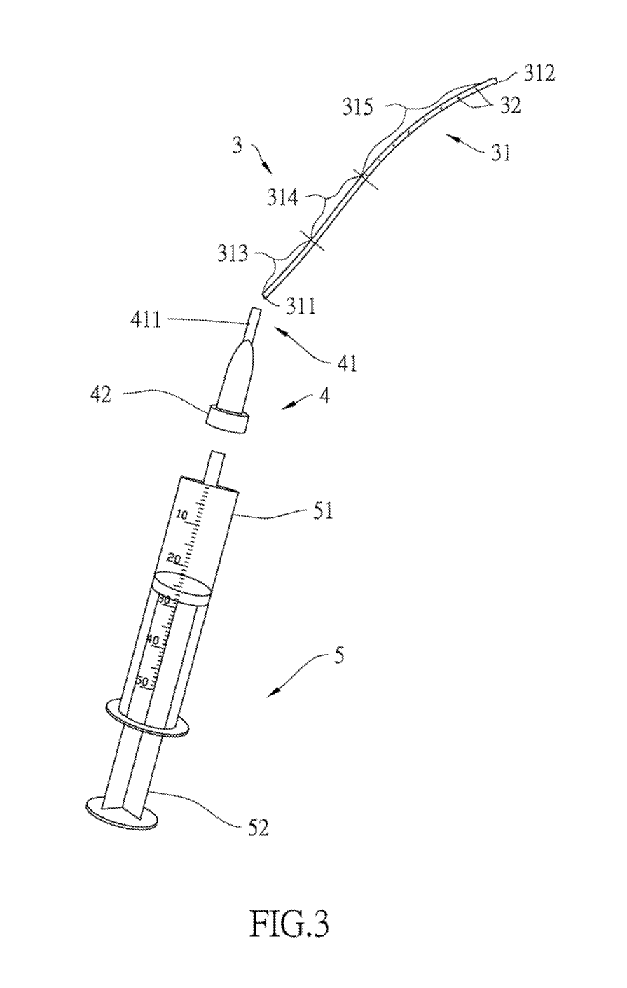

Please refer to FIG. 3. FIG. 3 is a perspective view of a nasal flushing device according to a first embodiment of the present invention. The nasal flushing device comprises a catheter unit 3, a connecting unit 4, and an injection unit 5.

The catheter unit 3 has a catheter body 31 and a plurality of apertures 32 formed on the catheter body 31. The catheter body 31 has an open end 311 and a closed end 312 opposite the open end 311. The closed end 312 can be straight or curved.

The catheter body 31 from the open end 311 to the closed end 312 is divided into a connecting section 313, a reaching section 314 and a flushing section 315. The connecting section 313 has a length from 1 cm to 5 cm, the reaching section 314 has a length from 1 cm to 5 cm, and the flushing section 315 has a length from 3 cm to 10 cm. The plurality of apertures 32 on the catheter body 31 is disposed on the flushing section 315. The catheter body 31 is made of silicon, latex, thermoplastic elastomer or other soft and elastic material. The consistency of the catheter is so soft that it cannot sustain its own weight on upright position. The soft consistency renders the catheter to be easily navigated along the tortuous nasal cavity without hurting the patient. Since average nasal meatus of patient with chronic sinusitis is not wider than 3 mm, the diameter of the catheter body 31 is not larger than 3 mm.

The connecting unit 4 has a connecting portion 41 and an assembling portion 42. The connecting tube 411 of the connecting portion 41 is for connecting the open end 311 of the catheter, while the assembling portion 42 is for connecting the injection unit.

The injection unit 5 has a detachable syringe 51 and a plunger 52 provided in the syringe 51. The syringe 51 and the plunger 52 are used for compressing the fluid for ejecting spouts.

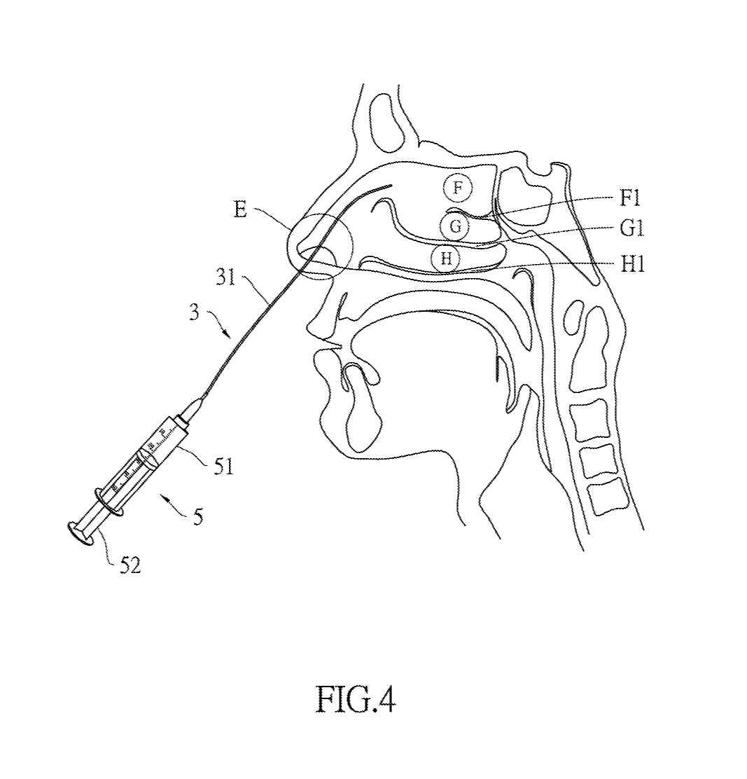

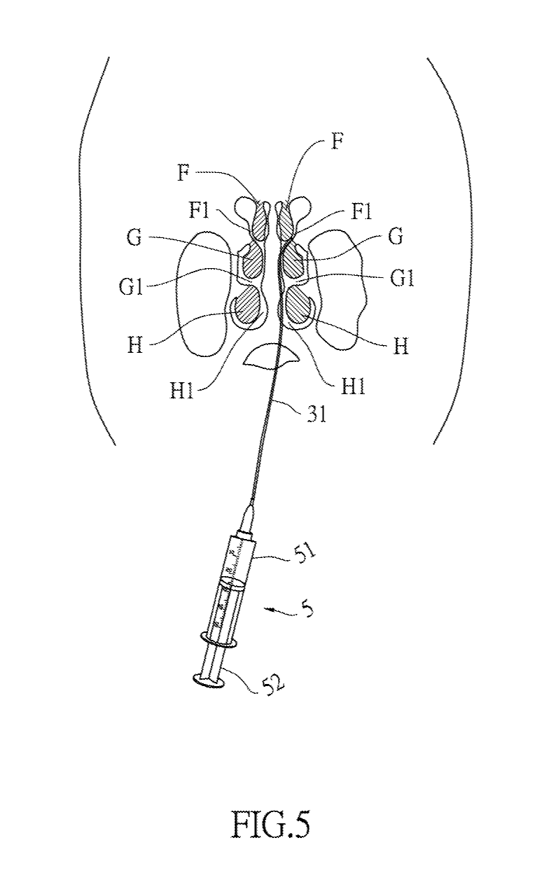

Please refer to FIG. 4 and FIG. 5 together. As showing the drawings, the nasal cavity on each side has a nasal vestibule E, a upper turbinate F, a middle turbinate G, an inferior turbinate H, an upper nasal meatus F1, a middle nasal meatus G1 and a lower nasal meatus H1. The nasal vestibule usually is about 3 cm long, the upper nasal meatus F1, the middle nasal meatus G1 and the lower nasal meatus H1 are usually about 5 cm in length. Therefore, in the first embodiment, a length of the flushing section 315 of the catheter body 31 is 5 cm, the plurality of apertures 32 are arranged apart every 0.1 cm to 1 cm circumferentially around the surface of the catheter body 31. A total length of the catheter body 31 is about 10 cm to 17 cm, the length of the flushing section 315 is 3 cm to 7 cm, the length of the reaching section 314 in the nasal vestibule E is 2 cm to 5 cm, the remaining length of the connecting section 313 is 3 cm to 5 cm, which can be held by the user and provide further reaching length. The length of 17 cm is for reaching nasopharynx for cleansing the mucus or crust.

For actual application, the patient inserts the flushing section 315 of the catheter body 31 into the upper nasal meatus F1 or the middle nasal meatus G1, connects the catheter to a syringe 51 pre-filled with flushing fluid, then pushes the plunger 52 to push the flushing fluid in the syringe 51, so that multiple thin spouts with ejecting angles nearly perpendicular to the catheter body 31 spur out from the aperture 32 on the flushing section 315 of the catheter body 31. With ejecting angles nearly perpendicular to the catheter body 31, the catheter body 31 is free from counterforce movement so that it can maintain in a stable location during the injection and ensures the irrigation effect.

Since the flushing section 315 is configured to be disposed in the upper nasal meatus F1 of the nasal cavity, the flushing fluid can directly rinse the mucus or crust built up in the upper nasal meatus F1. Similarly, when the flushing section 315 is configured to be disposed in the middle nasal meatus G1 or the lower nasal meatus H1 the flushing fluid can directly rinse the mucus or crust built up in the middle nasal meatus G1 and the lower nasal meatus H1.

Moreover, since the catheter can eject multiple thin spouts up to 90 cm in length, even though the catheter body 3 is not inserted into roof of the upper nasal meatus F1, the spouts are capable of rinsing most area of upper meatus. The spouts might be strong enough to pass through thin gap into deeper area for a better cleaning result. Worth of mention, the pressure is averaged by the numerous spouts so that they would not damage the mucosa.

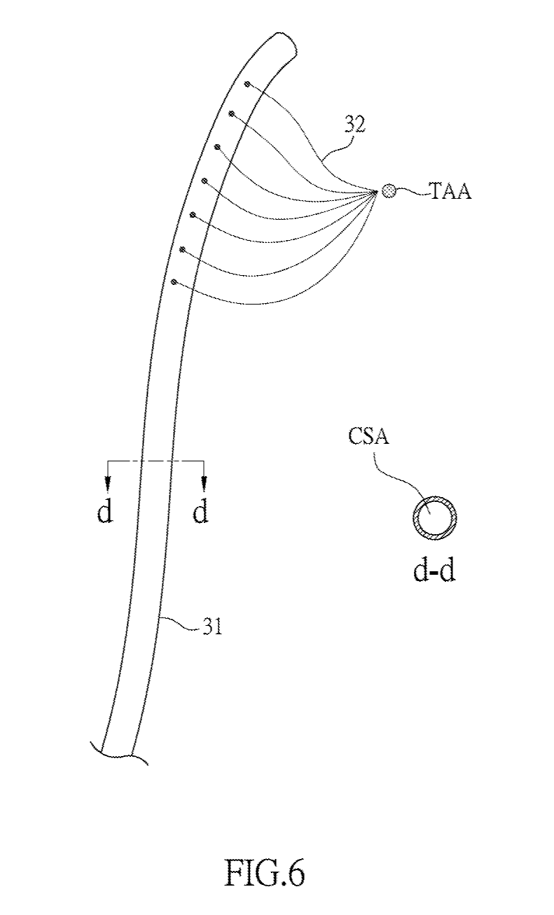

Please refer to FIG. 6, the total area of apertures 32 is represented as TAA, and the cross section area of inner lumen of catheter body 31 is represented as CSA. Substantially even strength of multiple thin spouts is very important for good efficiency in cleansing the mucus and crust in nasal cavity and nasopharynx. Substantially even strength of multiple thin spouts can be carried out by a special design of smaller TAA than CSA of the catheter which can be explained as following:

In a catheter body 31 with a closed distal end and multiple apertures 32, the flow rate through the inner lumen is determined by the cross section area of inner lumen of the catheter body 31; while the flow rate through the apertures 32 is determined by the total area of apertures 32.

If the total area of apertures 32 is larger than the cross section area of catheter body 31, during injection of fluid, the flow rate through the inner lumen is smaller than the flow rate through the whole apertures, the pressure within the lumen would be released from the apertures 32. The pressure is higher in the proximal portion and lower in the distal portion of catheter body 31 due to resistance of the catheter lumen and release of pressure from the proximal apertures 32. When the injected fluid passes to the first aperture 32 and if the intraluminal pressure is higher than that outside of the catheter body 31, the injected fluid would pass out from the first proximal aperture 32 to produce a spout; the intraluminal pressure becomes lower distal to the first aperture 32 due to release of some pressure from the first aperture 32, if the intraluminal pressure is still higher than the pressure outside of the catheter body 31, the injected fluid would pass out from second apertures 32 to produce a second spout weaker than the first spout. As the injected fluid passes toward distal end of the catheter body 31, the intraluminal pressure becomes lower and lower due to the resistance of lumen of catheter body 31 and release of pressure from the proximal apertures 32, the spouts from the distal apertures 32 becomes weaker and weaker. The resulting multiple spouts are uneven in strength, stronger proximally and weaker distally. This condition happens in all conventional catheters having fluid delivery, suction or drainage function.

The function of fluid delivery is defined as the flow rate from the lumen through the apertures 32 to outside of the catheter within the body; the function of suction or drainage is defined as the flow rate from outside of the catheter within the body through the apertures into the lumen of the catheter by force (suction) or spontaneously (drainage). Since the flow rate through the lumen of catheter body 31 is limited by the cross section area of the catheter body 31, the only way to improve function of fluid delivery, suction or drainage is to increase the total area of apertures 32. Therefore, these catheters all have larger total area of apertures 32 than the cross section area of catheter body 31 according to the design concept for better function, the larger the total area of apertures 32, the better the desired function. For ensuring the function of fluid delivery, suction or drainage, the total area of apertures 32 should not be smaller than the cross section area of inner lumen of catheter body 31.

On the other hand, if the total area of apertures 32 is smaller than the cross section area of inner lumen of catheter body 31, the flow rate of catheter lumen would be larger than the flow rate through all the apertures 32; during injection, the inflow is larger than the outflow of fluid, the injected fluid can easily reach the distal closed end and reflect back to cause the so called back pressure, repeated reflecting back of pressure would cause intraluminal pressure accumulation and resulting in high pressure evenly distributed throughout the lumen, therefore substantially even strength multiple thin spouts can be produced. The more difference between cross section area of inner lumen and total area of apertures 32 of catheter body 31, the higher the accumulated pressure and the stronger the multiple thin spouts. The back pressure and accumulation of intraluminal pressure cannot happen in conventional catheters.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A nasal ejecting catheter comprises a catheter body and a connector which are connected to an injection unit for home remedy of nasal irrigation treatment, wherein
the catheter body is made of silicone, latex, thermoplastic elastomer or other soft and elastic material, comprising a closed end, an open end on the opposite side and a plurality of apertures formed near the closed end, the total area of apertures is smaller than the cross section area of inner lumen of the catheter.
